(19) United States
(12) Patent Application Publication
(10) Pub. No.: US 2026/0153401 A1
(43) Pub. Date: Jun. 4, 2026

(54) CONTAINER CLOSURE INTEGRITY TESTING METHOD AND SYSTEM

(71) Applicant: HOFFMANN-LA ROCHE INC., Little Falls, NJ (US)

(72) Inventors: Markus HEMMINGER, Basel (CH); Paolo MORONI, Basel (CH); Irwann LE BOUQUIN, Basel (CH); Christian Oliver MUELLER, Basel (CH); Carolin SCHRANK, Basel (CH); Finn Henrik STEINBERG, Basel (CH); Joel Kai STRUB, Basel (CH)

(21) Appl. No.: 18/865,603

(22) PCT Filed: Aug. 24, 2023
(86) PCT No.: PCT/EP2023/073206

§ 371 Date: Nov. 13, 2024

(30) Foreign Application Priority Data
Aug. 25, 2022 (EP) 22192050.7

(51) Int. Cl. G01M 3/22; A61M 5/315

(52) U.S. Cl. CPC G01M 3/227; A61M 5/31513; G01M 3/229; A61M 2209/02

(57) ABSTRACT

A container closure integrity (CCI) testing method for testing tightness of a stopper closure of a syringe (7), wherein the syringe has a syringe body (71) with a longitudinal axis, a hollow interior extending between an open first axial end and a second axial end with an orifice (712), and an elastic stopper (73) provided through the open first axial end into the hollow interior such that a chamber (74) is formed between the stopper (73) and the second axial end, comprises: providing the syringe (7) in a gas environment comprising a detection gas; moving the stopper (73) inside the hollow interior of the syringe body (71); and sensing for detection gas exiting the chamber (74) of the syringe (7) while moving the stopper (73) inside the hollow interior of the syringe body (71).

### TECHNICAL FIELD

The present invention relates to a container closure integrity (CCI) testing method, a CCI testing system, a validation process and a syringe surrogate. Such testing method and system can be used for testing tightness of a stopper closure of a syringe, wherein the syringe has a syringe body with a longitudinal axis, a hollow interior extending between an open first axial end and a second axial end with an orifice, and an elastic stopper provided through the open first axial end into the hollow interior such that a chamber is formed between the stopper and the second axial end.

### BACKGROUND ART

In connection with provision of pharmaceutical and other sensitive substances integrity of involved container and packages, in which the substances are arranged is of high importance. Thereby, integrity of a container or package generally indicates the ability of keeping a content or substance inside the respective container or package and of keeping detrimental environmental contaminants outside the respective container or package.

For example, integrity of containers can be affected by a leak in the container or package. Leaks are typically perceived as holes or cracks of a certain diameter and length. Leakage may be a measure of gas flow (in mass or volume or units) that passes through a leak path under specific conditions. Leakage of 1 [mbar×I/sec] is given when the pressure in a closed container of 1 liter rises or falls within 1 sec by 1 mbar.

Specific container integrity issues have to be considered when containers having movable closures are involved. In particular, syringes prefilled with a pharmaceutical substance typically have a plunger rod provided with a stopper which has to be movable upon administration in order to expel the pharmaceutical substance through a needle or orifice. In order to prevent unintended movement of the stopper, the plunger can be physically fixed during shipping and other handling of the syringe.

However, during assembly and shipping of syringes, stoppers may still undergo certain movements. For example, during assembly such as, particularly, assembly of a plunger rod the stopper may undergo axial and/or rotational movement which may induce deformation of the stopper potentially affecting tightness at the stopper interface. Or, during shipping the syringe can be exposed to varying pressure which may induce certain linear movement of the stopper. For example, when shipping syringes by airplanes, the syringes are exposed to lower pressure during flight and higher or ambient pressure before and after landing. Thereby, a pressure difference between the inside of the syringe and the outside of the syringe of about 500 mbar may occur. Since the interior of a syringe also contains a gas or air bubble, such pressure differences can induce repetitive movement of the stopper which may affect integrity of the syringe closure provided by the stopper.

In order to test integrity of syringes, an acknowledged procedure is to provide a culture medium in the syringes, to expose the syringes to specific conditions and to verify if microbiological contaminants grow in the culture medium. However, such microbiological test procedures often are comparably prone to errors, time consuming and difficult to reproduce.

Therefore, there is a need for a container closure integrity testing method and system allowing testing syringes in a comparably reliable, quick and reproducible manner.

### DISCLOSURE OF THE INVENTION

According to the invention this need is settled by a container closure integrity (CCI) method as it is defined by the features of independent claim 1, by a CCI system as it is defined by the features of independent claim 18 and by a validation process for validating a CCI testing method as it is defined by the features of independent claim 36. Preferred embodiments are subject of the dependent claims.

In one aspect, the invention is a CCI testing method for testing tightness of a stopper closure of a syringe, wherein the CCI testing method advantageously is an essentially fully automatic method not requiring any manual interaction. The syringe has a syringe body with a longitudinal axis, a hollow interior extending between an open first axial end and a second axial end with an orifice, and an elastic stopper provided through the open first axial end into the hollow interior such that a chamber is formed between the stopper and the second axial end. The CCI testing method comprises the steps of: (i) providing the syringe in a gas environment comprising a detection gas; (ii) moving the stopper inside the hollow interior of the syringe body; and (iii) sensing for detection gas exiting chamber while moving the stopper inside the hollow interior of the syringe body.

Moving the stopper inside the hollow interior of the syringe body relates to a relative movement between the stopper and the syringe body. Such relative movement can be achieved by moving the stopper and holding the syringe body. Since the syringe body can be more efficiently held and accurately guided in an automatic process, preferably the stopper is held and the syringe body is moved. Also, a combination of such relative movements is possible.

Automatic relative movement of the stopper and the syringe body allows for increasing accuracy, efficiency and reproducibility of the CCI testing method.

Step (i) can be embodied by exposing the syringe or at least a portion thereof including the first axial end and/or the second axial end of the syringe body to a gas flow comprising the detection gas or to a static gas including the detection gas at a predefined static or variable pressure. For being exposed to a gas flow, the first and/or second axial end can be connected to a gas supply, such as a tube or pipe, forwarding the detection gas. Thus, step (i) covers only the first axial end being exposed to the gas flow, e.g., by being connected to a gas supply, only the second axial being exposed to the gas flow, e.g., by being connected to a gas supply, or the first axial end as well as the second axial end both being exposed to the gas flow. In the latter case, either the first axial end or the second axial end advantageously are tightly closed or tightly connected to other means such as a gas detector or the like.

The orifice of the syringe body of the syringe can be embodied as or equipped with a connection structure to be mounted to a needle such as a Luer lock structure or the like. Alternatively, it can be provided with a staked-in needle. Or, it can be embodied as spout.

The CCI testing method according to the invention allows for efficiently testing if and to what extent gas exiting the orifice includes detection gas present at the first axial end of the syringe body or exiting the open first axial end of the syringe body includes detection gas. From this it can be concluded if gas can pass the stopper and get into the chamber of the syringe. If such gas passage is possible to a predefined extent, it can be an indication that the closure provided by the stopper is not sufficiently tight to assure container closure integrity and sterility of the syringe content.

Moreover, by moving the stopper during step (iii) it can be achieved that container closure integrity is dynamically tested. This can be of particular importance, since it may be that the stopper is sufficiently tight before and after movement but not during movement. For example, while being moved, the elastic stopper may deform which can temporarily affect tightness of the closure.

Thus the CCI testing method according to the invention allows testing syringes in a comparably reliable, quick and reproducible manner.

Preferably, moving the stopper comprises axially moving the stopper in the hollow interior of the syringe body.

The term “axially moving” in this context relates to a movement along the longitudinal axis of the syringe body. The stopper can be axially moved in the syringe body by actively moving the stopper while holding the syringe body, by actively moving the syringe body while holding the stopper, or by a combination thereof.

Such axial movement of the stopper allows to mimic movements induced by pressure differences between the environment of the syringe and the chamber or induced by physical means, e.g. during assembly of a plunger rod. E.g., for assembly some plunger rods have to be coupled with the stopper which may lead to stopper forward movement and after coupling the stopper may move back again. Or other plunger rods have a thread which is screwed into the stopper cavity, which can lead to rotation of the stopper and in some cases also to axial stopper movement. Since the axial movements are induced while the detection gas is provided, a potential leakage and dimension thereof can be detected by measuring the detection gas exiting the orifice or exiting the open first end of the syringe body. The effects of an eventual deformation of the stopper during axial movement are included.

Thereby, axially moving the stopper preferably comprises forwarding the stopper towards the orifice and retracting the stopper towards the first axial end of the syringe body. Such back and forth movement of the stopper allows for mimicking repeatedly changing pressure differences between inside and outside of the syringe as it may occur, e.g., in an airplane when repeated taking off and landing is involved.

Thereby, a distance between a maximally forwarded position of the stopper and a maximally retracted position of the stopper preferably is in a range of about 15 mm to about 25 mm, in a range of about 10 mm to about 20 mm, or in range of about 5 mm to about 15 mm. Like this, various dimensions of syringes can be mimicked.

A speed of axially moving the stopper inside the hollow interior preferably is in a range of about 4 mm per minute to about 15 mm per minute, about 9.6 mm per minute, in range of about 15 mm per minute to about 25 mm per minute, or about 20 mm per minute.

Preferably, moving the stopper comprises rotating the stopper inside the hollow interior about the longitudinal axis of the syringe body.

The stopper can be rotated in the syringe body by actively rotating the stopper while holding the syringe body, by actively rotating the syringe body while holding the stopper, or by a combination thereof.

Such rotational movement of the stopper allows to efficiently mimic stopper deformations which may be induced by physical means, e.g. during assembly of a plunger rod. E.g., for assembly some plunger rods have a thread screwed into the stopper cavity, which can lead to rotation of the stopper. Thus, sealing issues during assembly of the syringe may be identified. The rotational movement can be the sole motion of the stopper, or, advantageously, a motion sequentially or simultaneously combined with the axial movement described above, Thereby, rotating the stopper inside the hollow interior of the syringe body preferably comprises a rotation of the stopper relative to the syringe body by about 180° or more, by about 360° or more, by about 720° or more, or by about 900° or more. Rotations of such extent allow for appropriately mimicking the situation potentially occurring. Like this, tightness of the stopper closure can be verified and approved for extreme conditions.

A speed of rotating the stopper inside the hollow interior of the syringe body preferably is about 700° per minute or more, about 5'000° per minute or more, about 20'000° per minute or more, or about 28'000° per minute or more. Such rotational velocity also allows for verifying and approving comparably appropriate conditions.

The CCI testing method may involve only axial stopper movement, only rotational stopper movement, a sequential combination of both or simultaneous axial and rotational stopper movements. However, moving the stopper inside the hollow interior of the syringe body preferably comprises at least one cycle of rotating the stopper and plural cycles of axially moving the stopper. Such multi cycle test protocol allows for an efficient and reliable approval of the tightness of the stopper closure.

Preferably, the detection gas comprises or is Helium. Helium is a particularly suitable detection gas medium as it consists of small atoms, is comparably easy to detect and is comparably economic.

The gas environment can provide the detection gas at any suitable pressure, i.e. any suitable underpressure or overpressure. However, preferably, the gas environment comprises the detection gas at a pressure of about 1 bar. Such pressure allows to mimic ambient conditions.

The syringe may be a plastic syringe such as, e.g., a polymeric syringe. Preferably, the syringe is a glass syringe having a nominal volume of 0.5 milliliter, 1 milliliter, 2.25 milliliter, 5 milliliter or 10 milliliter. Particularly, the nominal volume advantageously is between 0.5 milliliter and 10 milliliter. By stopper movement parameters as described above are involved, syringes of these dimensions may efficiently be verified and approved.

Preferably, providing the syringe in the gas environment comprises vertically positioning the syringe such that the orifice is upwardly or downwardly oriented and wherein the syringe is positioned in the upright position when moving the stopper inside the hollow interior of the syringe body and sensing for the detection gas. Such vertical arrangement allows for an efficient handling and verification or testing.

The sensing of the detection can be performed at any suitable location or portion of the syringe. In one preferred embodiment, the sensing for detection gas exiting the chamber comprises sensing for detection gas exiting the orifice of the syringe body while moving the stopper inside the hollow interior of the syringe body. In another preferred embodiment, the sensing for detection gas exiting the chamber comprises sensing for detection gas exiting the open first axial end of the syringe body while moving the stopper inside the hollow interior of the syringe body. Such sensing allows for a particular efficient sensing.

Preferably, the CCI testing method involves steps of automatically monitoring and recording movements of the stopper inside the hollow interior of the syringe body and sensed detection gas. By such monitoring and recording, information about stopper movement and detection gas leakage can be efficiently gathered. Like this, it is possible to evaluate at what point in time and/or by which movements leakage occurs. Such information may help to improve properties of the container closure in order to achieve appropriate tightness.

In the CCI testing method, the provision of the syringe in the gas environment can comprise non-tightly connecting a gas supply, e.g., a specific gas chamber, to the syringe body. In particular, such non-tight connection can be designed to allow leakage of the detection gas. Like this, when having a constant supply of detection gas to the syringe, the detection gas concentration inside the syringe can be held constant during relative stopper movement, since excess detection gas can bypass and flow into the environment. Thus, the detection gas concentration and the ambient pressure in the barrel can remain constant during CCI testing.

In an embodiment, the CCI testing method comprises a step of preparing the syringe prior providing the syringe in the gas environment.

Thereby, such preparation can include impairing the stopper of the syringe preferably before providing the stopper provided through the open first axial end into the hollow interior of the syringe body. For example, in case that the stopper comprises a plurality of radially extending sealing lips or sealing rings, such impairing may be implemented by destroying some of the sealing lips or rings, preferably, all sealing lips or rings except one.

Alternatively or additionally, such preparation includes choosing a syringe body having a specific inner diameter. In particular, due to clearances in manufacturing syringe bodies usually have range of accepted inner diameters wherein the specific inner diameter may by a maximum inner diameter accepted, e.g., in accordance with any legal provision or practical specification, or even a slightly larger inner diameter.

By preparing the stopper and/or the syringe body as described hereinbefore, an efficient robustness testing can be achieved. In particular, it can be demonstrated that the detection gas such as Helium cannot traverse any of the sealing lips or rings during stopper movement. This allows to conclude that bioburden such as bacteria also cannot traverse the sealing lip(s), since bacteria are typically significantly bigger than detection gas or Helium atoms.

In another aspect, the invention is a container closure integrity (CCI) testing system for testing tightness of a stopper closure of a syringe, wherein the syringe has a syringe body with a longitudinal axis, a hollow interior extending between an open first axial end and a second axial end with an orifice, and an elastic stopper provided through the open first axial end into the hollow interior such that a chamber is formed between the stopper and the second axial end. The CCI testing system comprises a detection gas reservoir configured to enclose a detection gas; a detection gas sensor; a processing unit having a gas chamber, a positioning structure and a movement structure; and a connector arrangement. The gas chamber of the processing unit is connected to the detection gas reservoir and configured to house detection gas provided by the detection gas reservoir. The positioning structure of the processing unit is configured to hold the syringe such that at least one of the open first axial end of the syringe body and the orifice of the second axial end of the syringe body is arranged in the gas chamber. The movement structure of the processing unit is configured to move the stopper in the hollow interior of the syringe body when the syringe is held by the positioning structure of the processing unit. The connector arrangement is configured to connect the other one of the open first axial end of the syringe body and the orifice of the second axial end of the syringe body to the detection gas sensor when the syringe is held by the positioning structure of the processing unit. The detection gas sensor is configured to sense for detection gas exiting the chamber of the syringe while moving the stopper in the hollow interior of the syringe body.

If the one of the open first axial end of the syringe body and the orifice of the second axial end of the syringe body is the open first axial end of the syringe body, the other one of the open first axial end of the syringe body and the orifice of the second axial end of the syringe body is the orifice of the second axial end of the syringe body. Vice versa, if the one of the open first axial end of the syringe body and the orifice of the second axial end of the syringe body is the orifice of the second axial end of the syringe body, the other one of the open first axial end of the syringe body and the orifice of the second axial end of the syringe body is the open first axial end of the syringe body.

The gas chamber of the processing unit can be embodied in any manner allowing to provide the syringe in a gas environment comprising the detection gas. For example, the gas chamber can be embodied by a rigid housing having an interior provided with the detection gas. Or, the gas chamber can be embodied as a tube or pipe connected or mounted to the syringe.

The CCI testing system according to the invention and its preferred embodiments described below allow to achieve the effects and benefits of the CCI testing method according to the invention and its preferred embodiments described above in an automatic manner. In particular, the CCI testing system can be configured to implement and automatically perform the CCI testing method.

Preferably, the CCI testing system comprises a control unit connected to the detection gas sensor and the processing unit. Such control unit allows for sophisticatedly operate the system as well as to collect and evaluate data produced in operation of the system.

The control unit can be embodied by a computer. The term “computer” in this connection can relate to any suitable computing device such as laptop computer, a desktop computer, a server computer, a tablet, a smartphone. The term covers single devices as well as combined devices. A computer can, for example, be a distributed system, such as a cloud solution, performing different tasks at different locations. A computer typically involves a processor or central processing unit (CPU), a permanent data storage having a recording media such as a hard disk, a flash memory or the like, a random access memory (RAM), a read only memory (ROM), a communication adapter such as an universal serial bus (USB) adapter, a local area network (LAN) adapter, a wireless LAN (WLAN) adapter, a Bluetooth adapter or the like, and a user interface such as a keyboard, a mouse, a touch screen, a screen, a microphone, a speaker or the like. Computers can be embodied with a broad variety of components as the components listed here.

The control unit preferably is configured to evaluate detection gas signals provided by the detection gas sensor. The term “signal” in this connection may particularly relate to a data or similar signal embodied as an electromagnetic signal such as an electrical voltage, radio wave, microwave, infrared signal or the like which can be physically transferred over a point-to-point or point-to-multipoint communication channel. Such channels may be copper wires, optical fibers, wireless communication channels, storage media and computer buses. The data signal can represent specific data particularly organized in accordance with a specific protocol such as, for example, the protocols mentioned above. The sensor data itself can be a digital bit stream or the like which represents physical and/or logical conditions and changes or the like. It can particularly be in a format accessible and evaluatable by the control unit.

Preferably, the movement structure of the processing unit is configured to axially move the stopper inside the hollow interior of the syringe body. The movement structure can be configured to actively move the stopper while the syringe body is held, to actively move the syringe body while the stopper is held, or by a combination thereof.

Thereby, the control unit preferably is configured to operate the movement structure of the processing unit such that a speed of axially moving the stopper inside the hollow interior of the syringe body is in a range of about 5 mm per minute to about 15 mm per minute, about 9.6 mm per minute, in range of about 15 mm per minute to about 25 mm per minute, or about 20 mm per minute.

The movement structure preferably is configured to axially move the stopper by forwarding the stopper towards the orifice and retracting the stopper towards the first axial end of the syringe body.

The control unit preferably is configured to operate the movement structure of the processing unit such that a distance between a maximally forwarded position of the stopper and a maximally retracted position of the stopper is in a range of about 15 mm to about 25 mm, in a range of about 10 mm to about 20 mm, or in range of about 5 mm to about 15 mm.

Preferably, the movement structure of the processing unit is configured to rotate the stopper inside the hollow interior about the axis of the syringe body. The movement structure can be configured to actively rotate the stopper while the syringe body is held, by actively rotate the syringe body while the stopper is held, or by a combination thereof.

Thereby, the control unit preferably is configured to operate the movement structure of the processing unit such that the stopper is rotated relative to the syringe body by about 360° or more, by about 720° or more, or about 900° or more.

The control unit preferably is configured to operate the movement structure of the processing unit such that a speed of rotating the stopper inside the hollow interior of the syringe body is about 700° per minute or more, about 5'000° per minute or more, about 20'000° per minute or more, or about 28'000° per minute.

The control unit preferably is configured to operate the movement structure of the processing unit such that moving the stopper comprises at least on cycle of rotating the stopper and plural cycles of axially moving the stopper.

Preferably, the detection gas comprises or is helium.

Preferably, the syringe is a glass syringe having a nominal volume of 0.5 microliter, 1 microliter, 2.25 microliter, 5 milliliter or 10 milliliter.

Preferably, the positioning structure of the processing unit is configured to vertically position the syringe such that the orifice is upwardly oriented.

Preferably, the detection gas reservoir and the gas chamber of the processing unit are configured to provide the detection gas at a pressure of about 1 bar in the gas chamber of the processing unit.

Preferably, the control unit is configured to automatically monitor and record movements of the stopper inside the hollow interior of the syringe body and sensed detection gas.

Preferably, the movement structure of the processing unit is configured to automatically move the stopper in the hollow interior of the syringe body when the syringe is held by the positioning structure of the processing unit. Automatic relative movement of the stopper and the syringe body allows for increasing accuracy, efficiency and reproducibility.

Preferably, the positioning structure of the processing unit is configured to non-tightly connect the one of the open first axial end of the syringe body and the orifice of the second axial end of the syringe body to the gas chamber. In particular, such non-tight connection can be designed to allow leakage of the detection gas. Like this, when having a constant supply of detection gas into the syringe, the detection gas concentration inside the syringe can be held constant during relative stopper movement, since excess detection gas can bypass and flow into the environment. Thus, the detection gas concentration and the ambient pressure in the barrel can remain constant during CCI testing.

In an embodiment, the stopper of the syringe is impaired. In case that the stopper comprises a plurality of radially extending sealing lips or sealing rings, such impairing may be implemented by destroying some of the sealing lips or rings, preferably, all sealing lips or rings except one.

Alternatively or additionally, the syringe body can be chosen to have a specific inner diameter. In particular, due to clearances in manufacturing syringe bodies usually have range of accepted inner diameters wherein the specific inner diameter may by a maximum inner diameter accepted, e.g., in accordance with any legal provision or practical specification, or even a slightly larger inner diameter.

In a further other aspect, the invention is a validation process for validating a CCI testing method as described above and/or a CCI testing system as described above. The validation process comprises the steps of: (i) obtaining a syringe surrogate having a surrogate body and a micro-capillary, wherein the syringe body has a longitudinal axis, a hollow interior extending between an open first axial end and a second axial end with an orifice, and wherein the micro-capillary is arranged in the orifice of the syringe body; (ii) providing the syringe surrogate in a gas environment comprising a detection gas; and (iii) sensing for the detection gas exiting the chamber of the syringe.

By means of the validation process according to the invention, sensitivity and, thus, appropriateness of the CCI testing method and system can be verified and validated. In particular, the syringe surrogate allows for mimicking a leaking syringe or, more specifically, a syringe having a leaking stopper closure. The validation process further allows to validate the CCI testing method and/or CCI testing system in accordance with an accepted standard such as in accordance with chapter <1207> “Containter-Closure Integrity Examination” of the Good Manufacturing Practice (GMP) widely applied in drug substance manufacturing.

In another further aspect, the invention is a syringe surrogate for being used in a validation process as described above. The syringe surrogate comprises a surrogate body and a micro-capillary. The syringe body has a longitudinal axis, a hollow interior extending between an open first axial end and a second axial end with an orifice. The micro-capillary is arranged in the orifice of the syringe body.

The term “micro-capillary” as used herein relates to microtubes, microcannula or micropipettes suitable for simulating single-orifice defects. The micro-capillaries may be formed of glass or any appropriate plastic or other material and can have a diameter in the range of approximately 0.1 μm to approximately 500 μm or, more specifically, in the range of approximately 2 μm to approximately 9 μm. A diameter up to about 10 μm or 15 μm may be appropriate for helium leakage testing. A diameter up to about 30 μm may be appropriate for vacuum decay or pressure decay testing micro-capillaries are usually employed as a substitute for smaller-bore, shorter-length leak path when performing tests that rely on gas flow measurements.

The syringe surrogate according to the invention allows for implementing an accurate validation process. In particular, by means of the micro-capillary a sufficiently small leakage can be simulated which allows to precisely validate CCI testing methods and systems.

Preferably, the surrogate body is made of a metal, particularly, of stainless steel. Such embodiment allows for a robust implementation. Thereby, it can be excluded that any leakage other than the simulated leakage occurs.

Preferably, the micro-capillary is made of glass. Such glass micro-capillary allows to provide inertness such that it can be assured that any detection gas passing the micro-capillary is not affected.

Preferably, the micro-capillary is tightened to the orifice of the surrogate body.

### BRIEF DESCRIPTION OF THE DRAWINGS

The CCI testing method according to the invention and CCI testing system according to the invention as well as the validation process according to the invention are described in more detail hereinbelow by way of exemplary embodiments and with reference to the attached drawings, in which:

FIG. 1 shows schematic view of a CCI testing system according to the invention;

FIG. 2 shows a detail of a processing unit of the CCI testing system of FIG. 1;

FIG. 3 shows a syringe operated in a CCI testing method according to the invention using the CCI testing system of FIG. 1;

FIG. 4 shows a diagram illustrating movement of a stopper of the syringe operated in the method of FIG. 3; and

FIG. 5 shows a diagram illustrating leakage of the syringe operated in the method of FIG. 3.

### DESCRIPTION OF EMBODIMENTS

In the following description certain terms are used for reasons of convenience and are not intended to limit the invention. The terms “right”, “left”, “up”, “down”, “under” and “above” refer to directions in the figures. The terminology comprises the explicitly mentioned terms as well as their derivations and terms with a similar meaning. Also, spatially relative terms, such as “beneath”, “below”, “lower”, “above”, “upper”, “proximal”, “distal”, and the like, may be used to describe one element's or feature's relationship to another element or feature as illustrated in the figures. These spatially relative terms are intended to encompass different positions and orientations of the devices in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. The devices may be otherwise oriented (rotated 90 degrees or at other orientations), and the spatially relative descriptors used herein interpreted accordingly. Likewise, descriptions of movement along and around various axes include various special device positions and orientations.

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

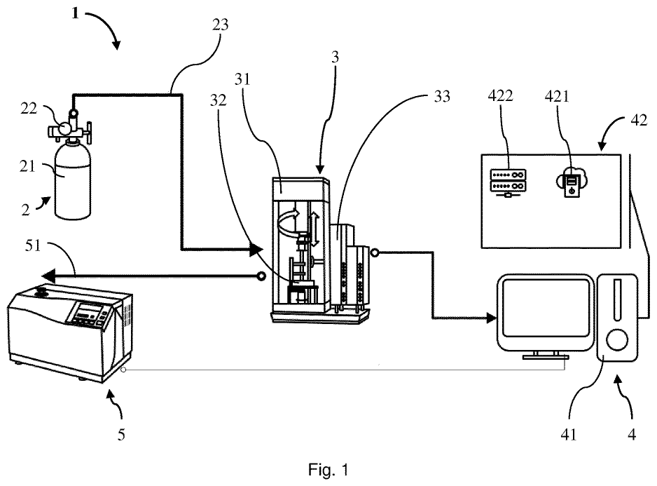

FIG. 1 shows an embodiment of a container closure integrity (CCI) testing system 1 according to the invention. The CCI testing system 1 comprises a detection gas reservoir 2, a processing unit 3, a control unit 4 and a detection gas sensor 5. The detection gas reservoir 2 has a Helium tank 21 configured to house Helium as detection gas. The detection gas reservoir 2 includes a pressure regulation unit 22 by which a pressure of the Helium supplied by the detection gas reservoir 2 is adjusted.

The processing unit 3 has a gas chamber 31, a positioning structure 32 and a movement structure 33. The movement structure 33 is equipped with a rotational drive and a linear drive. The gas chamber 31 of the processing unit 3 is connected to the detection gas reservoir 2 via a gas supply tube 23. It is further configured to house Helium as detection gas provided by the detection gas reservoir 2 through the gas supply tube 23.

The detection gas sensor 5 is connected to the processing unit 3 and, as described in more detail below, to a syringe 7 (not visible in FIG. 1) positioned inside the gas chamber 31 of the processing unit 3 via a leakage gas tube 51. The detection gas sensor 5 is configured to sense for Helium provided through the leakage gas tube 51.

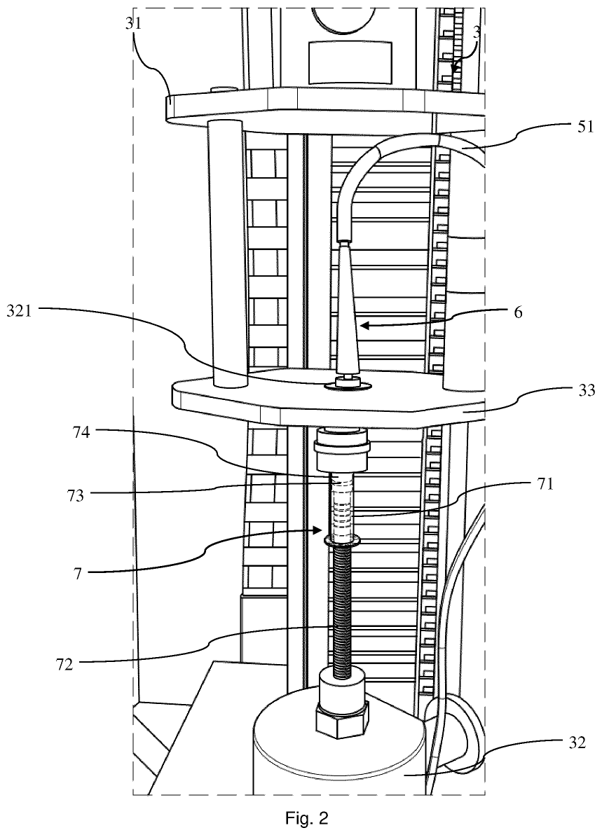

In FIG. 2, the positioning structure 32 of the processing unit 3 is shown in more detail. As can be seen, the positioning structure 32 comprises a syringe seat 321 receiving the syringe 7 in an upright or vertical position. The syringe 7 comprises a glass syringe body 71 with a longitudinal axis, a hollow interior extending between an open first axial end and a second axial end with an orifice 712 (not visible in FIG. 2), and a plunger 72 extending through the first axial end into the hollow interior. The plunger 72 is equipped with an elastic stopper 73 tightly closing the interior of the syringe body 71 such that a dosage chamber 74 is formed between the stopper 73 and the second axial end. The syringe 7 has a nominal volume of 2.25 ml. The syringe seat 321 of the positioning structure 32 is a multi-part construction configured to hold the syringe 7 such that the complete syringe body 71 including its first axial end is arranged in the gas chamber 31.

The CCI testing system 1 further comprises a connector arrangement 6 connecting the orifice of the syringe body 31 of the syringe 7 to the leakage gas tube 51. Thus, Helium exiting the syringe body 31 is provided to the detection gas sensor 5 via the leakage gas tube 51, where it is sensed and quantified.

Turning back to FIG. 1, the control unit 4 comprises a computer 41 executing a dedicated software. The computer 41 is connected to a network infrastructure 42 where it is connected to a backup server 421 and a data server 422. The control unit 4 is in communication with the processing unit 3 and the detection gas sensor 5. In particular, data generated by the detection gas sensor 5 is provided as data signals to the control unit 4 via a communication channel.

The movement structure 33 of the processing unit 3 is configured to move the stopper 73 in the hollow interior of the syringe body 71 when the syringe 7 is received in the syringe seat 321 of the positioning structure 32 of the processing unit 3. More specifically, the positioning structure 32 and the movement structure 33 are designed to hold the plunger 72 together with the stopper 73 in a fixed position and to move the syringe body 71 of the syringe 7 relative to the stopper 73.

The dedicated software configures the control unit 6 to apply an embodiment of a CCI testing method according to the invention. In particular, it is configured to operate the movement structure 33 such that the stopper 73 is linearly and rotationally moved relative the syringe body 71 in accordance with a specific test protocol. While moving the stopper 73 relative to the syringe body 71 and, thus, its hollow interior, the syringe 7 is exposed to the Helium inside the gas chamber 31 and the detection gas sensor 5 senses Helium exiting the orifice of the syringe 7.

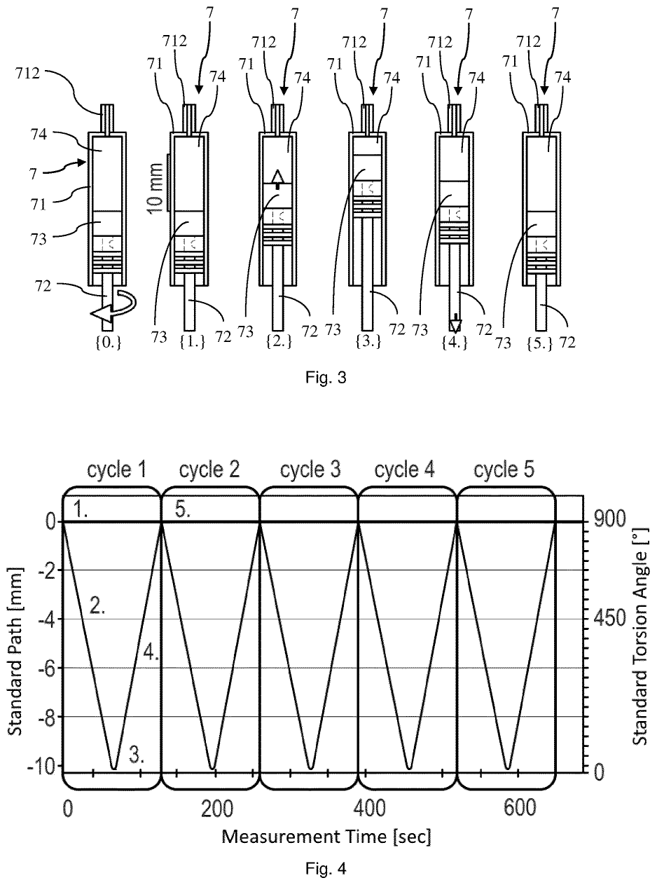

FIG. 3 shows the movements the syringe 7 undergoes when the control unit 6 operates the movement structure 33 be means of a schematic illustrations of the syringe 7. The left most illustration {0.} depicts a rotational movement of the stopper 73 relative to the syringe body 71. Thereby, the rotational drive of the movement structure 33 rotates the syringe body 71 by 900° about its longitudinal axis at a speed of 1.3 rotations per second. The other illustrations {1.} to {5.} depict an axial movement of the stopper 73 relative to the syringe body 71. Thereby, the linear drive of the movement structure 33 moves the syringe body 71 ten millimeter back and ten millimeter forth along the axis relative to the stopper 73. More specifically, illustrations {1.} to {3.} show the ten mm back movement of the syringe body 71 relative to the stopper 73 which induces a reduction of the volume of the dosage chamber 74. Illustrations {3.} to {5.} show the ten mm forth movement of the syringe body 71 relative to the stopper 73 which induces an increase of the volume of the dosage chamber 74. In total, the relative linear movement of the stopper 73 relative to the syringe body 71 is twenty mm per cycle.

In FIG. 4 shows the movements in accordance with the test protocol applied by means of the control unit 6. As can be seen, the test protocol induces a comparable fast first cycle of rotating the syringe body 71 relative to the stopper 73 as depicted in illustration {0.} of FIG. 3 followed by five cycles of linearly moving the syringe body 71 relative to the stopper 73 as depicted in illustrations {1.} to {5.} of FIG. 3. The number of the illustrations {1.} to {5.} of FIG. 3 are indicated in the first cycle of linear movement of FIG. 4. The complete test protocol runs for about 650 seconds.

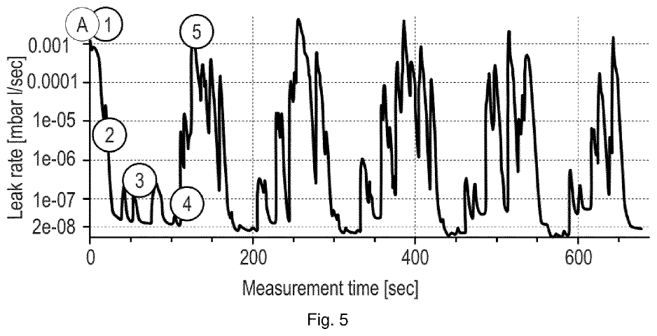

FIG. 5 shows the results of the detection gas sensor 5 measuring Helium exiting the orifice 712 of the syringe 7 during the complete test protocol applied by the control unit 6. More specifically, the control unit 6 connected to the detection gas sensor 5 obtains Helium leak rates during all cycles of the test protocol. Starting from the left, the movements depicted in illustrations {0.} to {5.} of FIG. 3 are indicated where they occur for the first time. As can be seen, in the example underlying the measurements shown in FIG. 5, the leak rate is above the Kirsch limit of 6×10⁻⁶ mbar I/sec in the rotational cycle and during linear back movement of the syringe body 71 relative to the stopper 73.

This description and the accompanying drawings that illustrate aspects and embodiments of the present invention should not be taken as limiting-the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the spirit and scope of this description and the claims. In some instances, well-known circuits, structures and techniques have not been shown in detail in order not to obscure the invention. Thus, it will be understood that changes and modifications may be made by those of ordinary skill within the scope and spirit of the following claims. In particular, the present invention covers further embodiments with any combination of features from different embodiments described above and below.

In particular, in an alternative embodiment similar to the embodiment described above in connection with FIGS. 1 and 2, element 51 of FIG. 2 is the gas supply tube 23 of FIG. 1 and element 6 is the gas chamber of the processing unit 3 connected to the detection gas reservoir 2 via a gas supply tube 23. In such embodiment, the detection gas is provided through the orifice of the syringe body into the chamber of the syringe. The detection gas is sensed after exiting the chamber of the syringe via the stopper out of the open first axial end of the syringe body.

The disclosure also covers all further features shown in the FIGS. individually although they may not have been described in the afore or following description. Also, single alternatives of the embodiments described in the figures and the description and single alternatives of features thereof can be disclaimed from the subject matter of the invention or from disclosed subject matter. The disclosure comprises subject matter consisting of the features defined in the claims or the exemplary embodiments as well as subject matter comprising said features.

Furthermore, in the claims the word “comprising” does not exclude other elements or steps, and the indefinite article “a” or “an” does not exclude a plurality. A single unit or step may fulfil the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively. The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

1. A container closure integrity (CCI) testing method for testing tightness of a stopper closure of a syringe, wherein the syringe has a syringe body with a longitudinal axis, a hollow interior extending between an open first axial end and a second axial end with an orifice, and an elastic stopper provided through the open first axial end into the hollow interior such that a chamber is formed between the stopper and the second axial end, comprising:
providing the syringe in a gas environment, wherein the gas environment comprises a detection gas;
moving the stopper inside the hollow interior of the syringe body; and
sensing for detection gas exiting the chamber while moving the stopper inside the hollow interior of the syringe body.

2. The CCI testing method of claim 1, wherein moving the stopper comprises axially moving the stopper in the hollow interior of the syringe body wherein axially moving the stopper preferably comprises forwarding the stopper towards the orifice and retracting the stopper towards the first axial end of the syringe body, wherein axially moving the stopper preferably comprises forwarding the stopper towards the orifice and retracting the stopper towards the first axial end of the syringe body, wherein a distance between a maximally forwarded position of the stopper and a maximally retracted position of the stopper preferably is in a range of about 15 mm to about 25 mm, in a range of about 10 mm to about 20 mm, or in range of about 5 mm to about 15 mm.

3. (canceled)

4. (canceled)

5. The CCI testing method of claim 2, wherein a speed of axially moving the stopper inside the hollow interior is in a range of about 4 mm per minute to about 15 mm per minute, about 9.6 mm per minute, in range of about 15 mm per minute to about 25 mm per minute, or about 20 mm per minute.

6. The CCI testing method of claim 1, wherein moving the stopper comprises rotating the stopper inside the hollow interior about the longitudinal axis of the syringe body.

7. The CCI testing method of claim 6, wherein rotating the stopper inside the hollow interior of the syringe body comprises a rotation of the stopper relative to the syringe body by about 180° or more, by about 360° or more, by about 720° or more, or by about 900° or more, and/or wherein a speed of rotating the stopper inside the hollow interior of the syringe body is about 700° per minute or more, about 5'000° per minute or more, about 20'000° per minute or more, or about 28'000°per minute or more.

8. (canceled)

9. The CCI testing method of claim 2, wherein moving the stopper comprises rotating the stopper inside the hollow interior about the longitudinal axis of the syringe body and wherein moving the stopper inside the hollow interior of the syringe body comprises at least one cycle of rotating the stopper and plural cycles of axially moving the stopper.

10. The CCI testing method of claim 1, wherein providing the syringe in the gas environment comprises vertically positioning the syringe such that the orifice is upwardly or downwardly oriented and wherein the syringe is positioned in the upright position when moving the stopper inside the hollow interior of the syringe body and sensing for the detection gas.

11. The CCI testing method of claim 1, wherein the sensing for detection gas exiting the chamber comprises sensing for detection gas exiting the orifice of the syringe body while moving the stopper inside the hollow interior of the syringe body.

12. The CCI testing method of claim 1, wherein sensing for detection gas exiting the chamber comprises sensing for detection gas exiting the open first axial end of the syringe body while moving the stopper inside the hollow interior of the syringe body.

13. The CCI testing method of claim 1, wherein the syringe is a glass syringe having a nominal volume of 0.5 milliliter, 1 milliliter, 2.25 milliliter, 5 milliliter or 10 milliliter, and/or wherein the syringe is provided in the gas environment at a pressure of about 1 bar, and/or wherein the detection gas comprises Helium.

14. (canceled)

15. (canceled)

16. The CCI testing method of claim 1, wherein moving the stopper inside the hollow interior of the syringe body comprises holding the stopper and moving the syringe body relative to the stopper.

17. The CCI testing method of claim 1, comprising automatically monitoring and recording movements of the stopper inside the hollow interior of the syringe body and sensed detection gas.

18. A container closure integrity (CCI) testing system for testing tightness of a stopper closure of a syringe, wherein the syringe has a syringe body with a longitudinal axis, a hollow interior extending between an open first axial end and a second axial end with an orifice, and an elastic stopper provided through the open first axial end into the hollow interior such that a chamber is formed between the stopper and the second axial end, the CCI testing system comprising:
a detection gas reservoir configured to enclose a detection gas;
a detection gas sensor;
a processing unit having a gas chamber, a positioning structure and a movement structure; and
a connector arrangement
wherein the gas chamber of the processing unit is connected to the detection gas reservoir and configured to house detection gas provided by the detection gas reservoir;
wherein the positioning structure of the processing unit is configured to hold the syringe such that one of the open first axial end of the syringe body and the orifice of the second axial end of the syringe body is arranged in the gas chamber;
wherein the movement structure of the processing unit is configured to move the stopper in the hollow interior of the syringe body when the syringe is held by the positioning structure of the processing unit;
wherein the connector arrangement is configured to connect the other one of the open first axial end of the syringe body and the orifice of the second axial end of the syringe body to the detection gas sensor when the syringe is held by the positioning structure of the processing unit; and
wherein the detection gas sensor is configured to sense for detection gas exiting the chamber of the syringe while moving the stopper in the hollow interior of the syringe body.

19. The CCI testing system of claim 18, comprising a control unit connected to the detection gas sensor and the processing unit, wherein the control unit preferably is configured to evaluate detection gas signals provided by the detection gas sensor.

20. (canceled)

21. The CCI testing system of claim 18, wherein the movement structure of the processing unit is configured to axially move the stopper inside the hollow interior of the syringe body, wherein the control unit preferably is configured to operate the movement structure of the processing unit such that a speed of axially moving the stopper inside the hollow interior of the syringe body is in a range of about 5 mm per minute to about 15 mm per minute, about 9.6 mm per minute, in range of about 15 mm per minute to about 25 mm per minute, or about 20 mm per minute.

22. (canceled)

23. The CCI testing system of claim 21, wherein the movement structure is configured to axially move the stopper by forwarding the stopper towards the orifice and retracting the stopper towards the first axial end of the syringe body, wherein the control unit preferably is configured to operate the movement structure of the processing unit such that a distance between a maximally forwarded position of the stopper and a maximally retracted position of the stopper is in a range of about 15 mm to about 25 mm, in a range of about 10 mm to about 20 mm, or in range of about 5 mm to about 15 mm.

24. (canceled)

25. The CCI testing system of claim 18, wherein the movement structure of the processing unit is configured to rotate the stopper inside the hollow interior about the axis of the syringe body, wherein the control unit preferably is configured to operate the movement structure of the processing unit such that the stopper is rotated relative to the syringe body by about 360° or more, by about 720° or more, or about 900° or more, and/or wherein the control unit preferably is configured to operate the movement structure of the processing unit such that a speed of rotating the stopper inside the hollow interior of the syringe body is about 700° per minute or more, about 5'000° per minute or more, about 20'000° per minute or more, or about 28'000° per minute, and/or wherein the control unit is configured to operate the movement structure of the processing unit such that moving the stopper comprises at least on cycle of rotating the stopper and plural cycles of axially moving the stopper.

26. (canceled)

27. (canceled)

28. (canceled)

29. The CCI testing system of claim 18, wherein the positioning structure of the processing unit is configured to vertically position the syringe such that the orifice is upwardly oriented, and/or
wherein the detection gas reservoir and the gas chamber of the processing unit are configured to provide the detection gas at a pressure of about 1 bar in the gas chamber of the processing unit, and/or
wherein the syringe is a glass syringe having a nominal volume of 0.5 microliter, 1 microliter, 2.25 microliter, 5 milliliter or 10 milliliter, and/or
wherein the detection gas comprises Helium.

30. (canceled)

31. (canceled)

32. (canceled)

33. The CCI testing system of claim 18, wherein the control unit is configured to automatically monitor and record movements of the stopper inside the hollow interior of the syringe body and sensed detection gas.

34. The CCI testing system of claim 18, wherein the movement structure of the processing unit is configured to automatically move the stopper in the hollow interior of the syringe body when the syringe is held by the positioning structure of the processing unit.

35. The CCI testing system of claim 18, wherein the positioning structure of the processing unit is configured to non-tightly connect the one of the open first axial end of the syringe body and the orifice of the second axial end of the syringe body to the gas chamber.

36. A validation process for validating a CCI testing method according to claim 1 and/or a CCI testing system according to claim 18, comprising:
obtaining a syringe surrogate having a surrogate body and a micro-capillary, wherein the syringe body has a longitudinal axis, a hollow interior extending between an open first axial end and a second axial end with an orifice, and wherein the micro-capillary is arranged in the orifice of the syringe body;
providing the syringe surrogate in a gas environment comprising a detection gas; and
sensing for the detection gas exiting the chamber of the syringe.

37. The validation process of claim 36, wherein the surrogate body of the syringe surrogate is made of a metal,
wherein the metal preferably is stainless steel, and/or
wherein the micro-capillary of the syringe surrogate preferably is made of glass, and/or
wherein the micro-capillary of the syringe surrogate is tightened to the orifice of the surrogate body.

38. (canceled)

39. (canceled)

40. (canceled)
